(19) United States
(12) Patent Application Publication
(10) Pub. No.: US 2007/0270817 A1
(43) Pub. Date: Nov. 22, 2007

(54) Connector apparatus

(75) Inventor: Alan Rezach, Atoka, TN (US)

Correspondence Address: WOODARD, EMHARDT, MORIARTY, MCNETT & HENRY LLP, 111 MONUMENT CIRCLE, SUITE 3700, INDIANAPOLIS, IN, 46204-5137, US

(73) Assignee: SDGI Holdings, Inc.

(21) Appl. No.: 11/409,773

(22) Filed: Apr. 24, 2006

(51) Int. Cl. A61F 2/30

(52) U.S. Cl. 606/61

(57) ABSTRACT

A connector apparatus is discussed for linking orthopedic elongated members, the apparatus having a connector body defining a first channel configured to receive a first elongate member and a second channel configured to receive a second elongate member. The channels are each defined by an inner contact surface configured to engage the corresponding elongate member. The first channel is U-shaped and opens at a top surface of the body. Additionally, the first channel includes an inner threaded portion configured to receive a first threaded retaining member to secure the first elongate member in the first channel. The second channel opens at a side surface of the body. Preferably, the channels open in substantially perpendicular directions. Additionally, the connector body defines a threaded through-hole, in communication with the second channel, configured to receive a second threaded retaining member to secure the second elongate member in the second channel.

The present disclosure broadly concerns spinal fixation systems and generally relates to a connector apparatus used to connect spinal rods. The apparatus can be useful for correction of spinal injuries or deformities.

### BACKGROUND

Several techniques and systems have been developed for use in correcting and stabilizing spinal curvatures, and for facilitating spinal fusion in the case of spinal disorders or degenerative conditions. In some systems, a pair of bendable rods may be longitudinally disposed adjacent the vertebral column and are fixed to various vertebrae along the length of the spine by way of a number of fixation elements, such as hooks and screws. In certain situations, it is desirable to supplement an existing spinal rod connected to the vertebral column with a new spinal rod, to add strength and stability to the fixation system.

Numerous spinal rod systems have been developed which provide transverse connectors for linking the adjacent spinal rods across the spinal midline to provide a rigid and stable construct. Such systems can present one or more difficulties for spinal surgeons. Many of the devices are high profile which increases soft tissue trauma and surgical complications. Moreover, it certain situations it is desirable to provide a transverse connection between adjacent spinal rods on the same side of the spinal midline.

Rigid transverse connections between spinal rods are beneficial because they restrict rod migration and increase construct stiffness. In many cases involving multi-level fusion of the spine, these features are essential while solid bone fusion is accomplished. In the post-operative period before fusion occurs, a significant amount of motion can occur between rods or other elongated members and other structure such as wires and hooks. That motion can, for example, allow a scoliotic correction to decrease or the pelvis to de-rotate towards a previous, deformed position. By providing a rigid transverse connection between two spinal rods, the loss of correction can be reduced and a stiffer construct can be created which may enhance the promotion of a solid fusion. A need remains for low profile devices which link adjacent spinal rods in a top-loading, top-tightening fashion with a minimum of components and steps, providing increased stability to the fixation system.

### BRIEF DESCRIPTION OF THE DRAWINGS

FIG. 1 is a side elevation view of a connector assembly according to an embodiment of the present application.

FIG. 2 is a top elevation view of a connector assembly according to an embodiment of the present application.

FIG. 3 is a cross-sectional view of the connector assembly of FIG. 2.

FIG. 4 is a perspective view of a connector assembly relative to vertebrae according to an embodiment of the present application.

FIG. 5 is another perspective view of a connector assembly relative to vertebrae according to an embodiment of the present application.

### DESCRIPTION OF THE ILLUSTRATED EMBODIMENTS

For the purpose of promoting an understanding of the principles of the disclosure, reference will now be made to the embodiments illustrated in the drawings and specific language will be used to describe the same. It will nevertheless be understood that no limitation of the scope of the claims is thereby intended, such alterations and further modifications in the illustrated device, and such further applications of the principles of the disclosure as illustrated therein, being contemplated as would normally occur to one skilled in the art to which the disclosure relates.

In certain embodiments of the present application, a connector apparatus for linking elongate members comprises a connector body defining a first channel configured to receive a first elongate member and a second channel configured to receive a second elongate member. The channels are each defined by an inner contact surface configured to engage the corresponding elongate member. The first channel is U-shaped and opens at a top surface of the body. Additionally, the first channel includes an inner threaded portion configured to receive a first threaded retaining member to secure the first elongate member in the first channel. The second channel opens at a side surface of the body. The channels may open in substantially perpendicular directions. Additionally, the connector body defines a threaded through-hole, in communication with the second channel, configured to receive a second threaded retaining member to secure the second elongate member in the second channel. The connector apparatus creates a low profile, side opening, top tightening fixation system, providing a stable, rigid system that sufficiently restricts movement and bending of the spinal rods and increases overall rigidity.

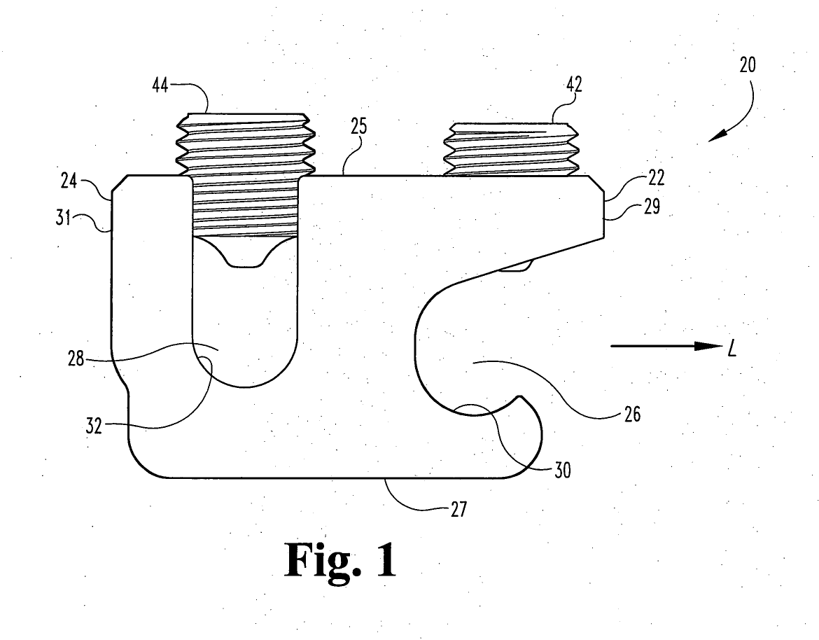

Referring generally to FIG. 1, there is shown an embodiment of a connector device 20 having a longitudinal axis L. Connector device 20, in that embodiment, has a first engagement portion 22 and a second engagement portion 24. Portion 22 defines a side-opening channel 26, and portion 24 defines a top-opening channel 28. Additionally, connector device 20 includes a top surface 25 opposite a bottom surface 27, both top and bottom surfaces generally parallel with longitudinal axis L, and side surfaces 29 and 31. Channel 26 includes an inner contact surface 30 and channel 28 includes an inner contact surface 32. Channels 26 and 28 are configured to receive elongate members, such as spinal rods. In the illustrated embodiment, channels 26 and 28 open in substantially perpendicular directions, with channel 26 opening toward side surface 29 and channel 28 opening toward top surface 25. Tightening members, such as screws 42 and 44, can be used to secure elongate members in channels 26 and 28.

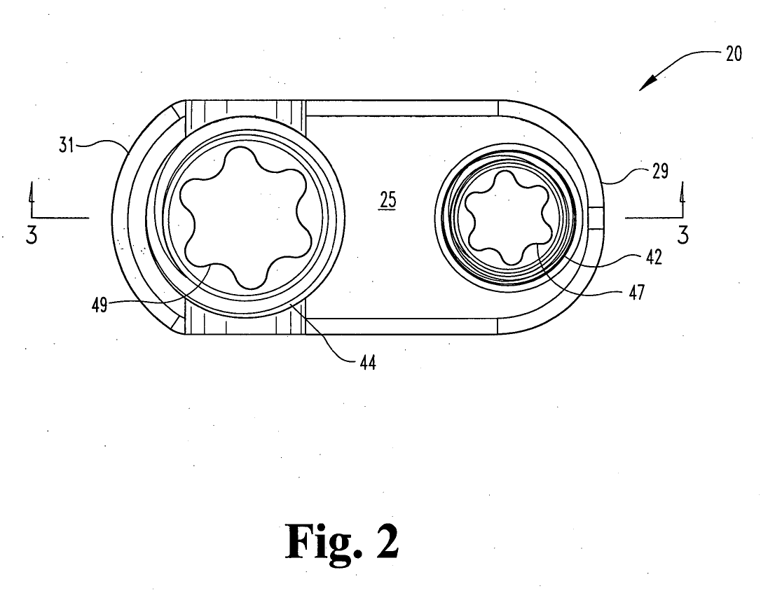

FIG. 2 illustrates a top view of connector device 20 with screws 42 and 44 positioned in engagement with connector device 20. In certain embodiments, screws 42 and 44 can be inserted to a position where the tops of the screws are below top surface 25. In such embodiments, screws 42 and 44 may rest essentially within connector device 20. In other embodiments, one or both of screws 42 and 44 could include an upper portion, such as a hexagonal head, that remains above top surface 25 of connector device 20. As one example, one or both of screws 42 and 44 could be break-off set screws having break-off top portions. It will be appreciated that screws 42 and 44 could be other appropriate types of screw, or could be replaced by other appropriate locking member(s).

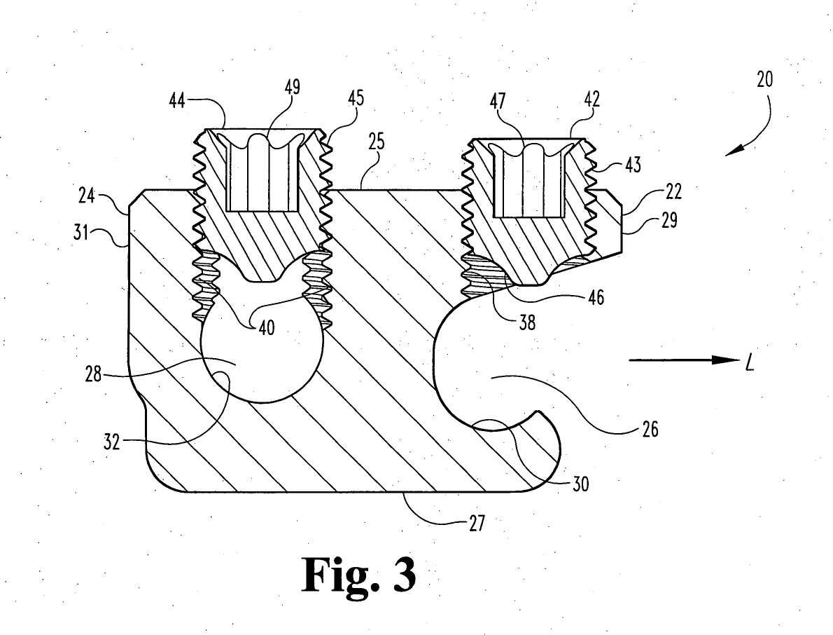

FIG. 3 is a cross-sectional view of connector device 20 taken along section lines 3-3 of FIG. 2. As illustrated, first engagement portion 22 defines a threaded through-hole 38 and second engagement portion 24 includes threaded portions 40. Through-hole 38 and threaded portions 40 are configured to engage with and receive tightening members, such as screws 42 and 44. Through-hole 38 and threaded portions 40 are aligned so that screws 42 and 44 advance in directions substantially perpendicular to top surface 25 and axis L of connector device 20. Through-hole 38 is in communication with channel 26. Screws 42 and 44 are operable to lock connector device 20 to elongate members such as spinal rods or bars, as further discussed below. Screws 42 and 44 provide a top tightening configuration, with the screws entering through top surface 25 of device 20 and being advanced through through-hole 38 and along threaded portions 40, respectively, to engage connector device 20 to elongate members.

The illustrated embodiment of screws 42 and 44 include threaded portions 43 and 45, respectively, which engage with threaded surfaces of through-hole 38 and threaded portions 40. Screw 42 includes a bearing surface 46 configured to contact and push a spinal rod into engagement with inner contact surface 30. Bearing surface 46 is shaped to conform to an outer surface of a rod positioned in channel 26. In some embodiments, bearing surface 46 is curved in a similar manner as the curved outer surface of a spinal rod. Additionally, screws 42 and 44 include internal, recessed hexagonal tops 47 and 49 to receive conventional driving tools. In other embodiments, other internal prints or external configurations could be used for accommodating gripping or driving tools. In the illustrated embodiment, through-hole 38 and screw 42 are at least partially offset from the positioning of a spinal rod in channel 26.

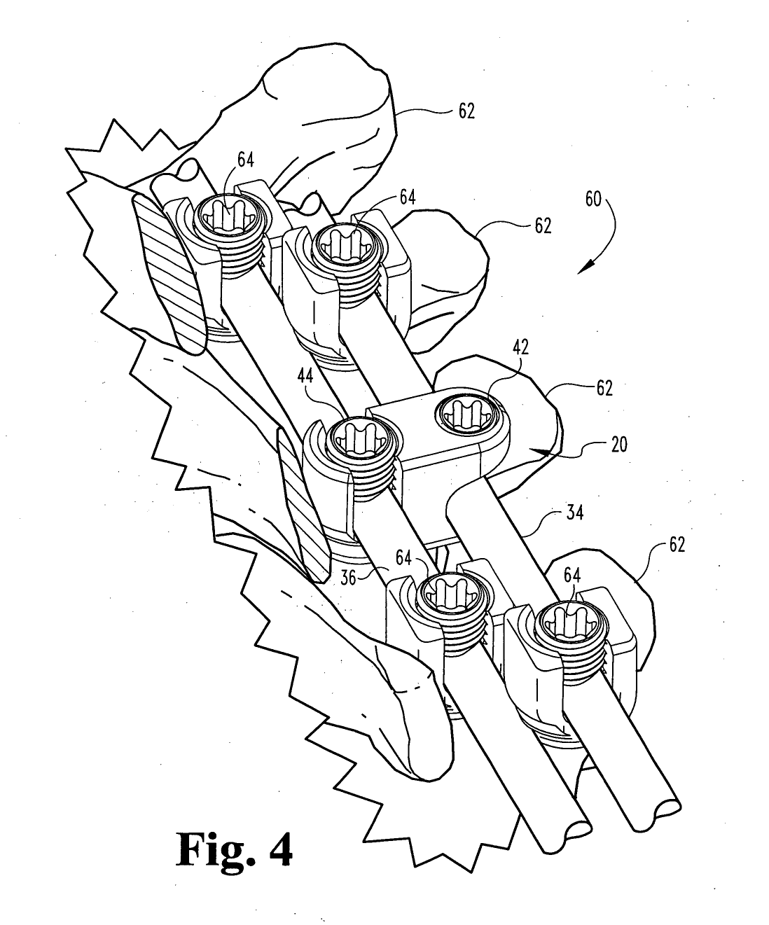

Referring generally to FIG. 4, there is shown a perspective view of an embodiment of connector device 20 relative to a section of the spine 60 including vertebrae 62. As illustrated, screws 42 and 44 can be advanced through through-hole 38 and along threaded portions 40, respectively, to engage connector device 20 to a first spinal rod 34 and a second spinal rod 36. In the illustrated embodiment, spinal rods 34 and 36 are connected to vertebrae 62 at various connection points. Spinal rods 34 and 36 can be connected to vertebrae 62 by pedicle screws 64 that are threaded into respective vertebrae 62, or by other such similar fixation elements.

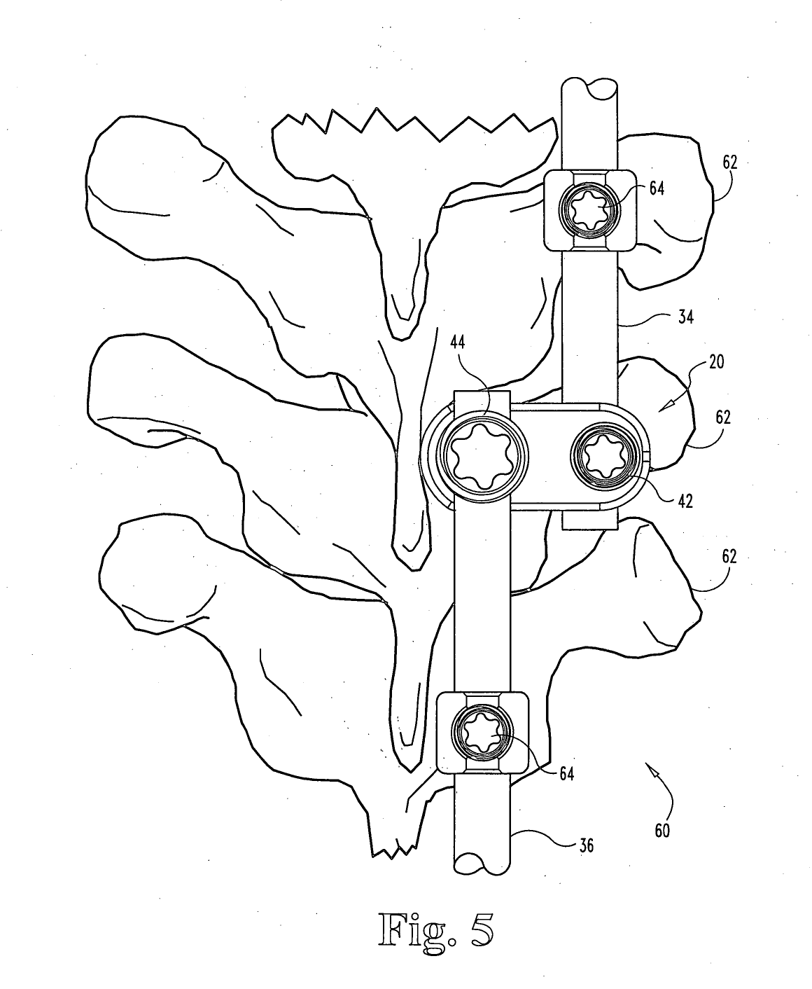

In certain embodiments, as illustrated, both spinal rods 34 and 36 are positioned on the same side of the spinal midline, or the spinous processes, of vertebrae 62. In other words, spinal rods 34 and 36 can both be positioned between one transverse process and the adjacent spinous process of each relevant vertebra 62. Positioning rods 34 and 36 in this fashion can be done in 5 several ways. Rods 34 and 36 can be placed simultaneously, providing a dual-rod construct along a portion of the spine (e.g. FIG. 4), or a construct in which one rod is attached to and extends along one set of vertebrae and is connected to another rod attached to another set of vertebrae (e.g. FIG. 5). Rods 34 and 36 may also be placed separately. For example, if rod 34 has been placed in a previous surgery, another rod 36 can be placed later in a revision surgery. Thus, in the latter situation, existing or previously-placed rod 34 can remain in place, without the necessity to remove tissue that has grown in contact with it or associated implants. Further, positioning rods 34 and 36 somewhat laterally, as shown, avoids the necessity to remove bone material of the spinous processes. In other embodiments, one spinal rod could be positioned on each side of the spinous processes and connector device 20 can cross the spinal midline.

FIG. 5 provides another perspective view of connector device 20 relative to vertebrae 62. In this embodiment, rods 34 and 36 are positioned on the same side of the spinous processes of the vertebrae. In the previous illustration, spinal rods 34 and 36 are connected to at least one common vertebra. FIG. 5 illustrates a type of “vertical connection” where the principal or only point of connection between spinal rods 34 and 36 is connector device 20. The configuration of connector device 20 allows for a sturdy, stable “vertical connection” with both spinal rods on the same side of the spinal midline.

The use of connector device 20 will be described in certain embodiments as follows, with particular reference to a spinal orthopedic procedure. It will be appreciated that other uses of connector 20 in other surgical procedures could be made.

Once an appropriate access to a surgical site is obtained, connector 20 can be inserted to the surgical site, and may be placed in a desired position at or adjacent certain vertebra(e) 62. In certain embodiments, a surgical procedure may be needed to revise a prior surgery. In such cases, spinal rod 36 may be an existing spinal rod that was previously connected to vertebrae 62 via pedicle screws 64, and spinal rod 34 is to be introduced to the surgical site and connected to vertebrae 62. Connector device 20 may be loaded onto spinal rod 36 from an underneath direction, with rod 36 being positioned in channel 28 at a desired position along rod 36. Thus, connector device 20 can be loaded onto spinal rod 36 from a position between rod 36 and a given vertebra 62 and/or other tissue material. Additionally, spinal rod 36 is loaded through an opening of channel 28 in top surface 25. Screw 44 is inserted into channel 28, so that threaded portion 45 engages with threaded portions 40. Screw 44 is advanced through channel 28 so as to bear against and push spinal rod 36 against inner contact surface 32. Screw 44 is sufficiently tightened to engage and lock connector device 20 to spinal rod 36.

Rod 34 can be pre-loaded into or otherwise connected to connector device 20 before engagement of connector device 20 to spinal rod 36, or rod 34 can be loaded into or otherwise connected to connector device 20 after engagement of connector device 20 to spinal rod 36. Spinal rod 34 is loaded into connector device 20 from a side direction, at a desired position along rod 34. Screw 42 is inserted into and advanced through through-hole 38 so as to bear against and push spinal rod 34 against inner contact surface 30. In the illustrated embodiment, bearing surface 46 of screw 42 contacts rod 34 to urge rod 34 against contact surface 30. Screw 42 is sufficiently tightened to engage connector device 20 to spinal rod 34. Final engagement is accomplished by tightening screws 42 and 44 against spinal rods 34 and 36, thereby locking the spinal rods laterally relative to each other. Spinal rod 34 can be connected to vertebrae 62 via pedicle screws 64. A vertebral fixation system involving connector device 20, spinal rods 34 and 36, and screws 42 and 44 is now in place, providing a rigid transverse connection between the adjacent spinal rods.

It will be appreciated that an existing rod (e.g. rod 36) can be received in channel 26 and a new rod (e.g. rod 34) can be received in channel 28. Thus, connector 20 can be maneuvered toward rod 36 from the side so that a portion of rod 36 enters channel 26 and is adjacent contact surface 30 of connector 20. Screw 42 can be threaded into connector 20 so as to loosely or tightly hold rod 36 in channel 26. If further adjustments of connector 20 with respect to rod 36 are expected or possible, then a loose holding of rod 36 can easily allow such adjustments, and tightening of screw 42 can occur after any final adjustments. Rod 34 can be placed in channel 28 either before or after connection of connector 20 to rod 36, and connector 20 (if loosely connected to rod 36) could be rotated or further maneuvered so that channel 28 is adjacent rod 34. Such rotation or maneuvering may be necessary if rod 34 has been fixed or otherwise connected to another implant or vertebra 62, and thus has less freedom of movement.

The above-described methods are useful both with the parallel dual-rod construct embodiment shown in FIG. 4, and with the approximately linear construct embodiment shown in FIG. 5. In the former, rods 34 and 36 are connected to one or more vertebrae 62 in common. In that situation, it may be difficult to insert a connector to provide lateral stabilization. Connector device 20, as noted above, provides for loading of one rod from the side of connector 20, and for loading of the other from the top of connector 20 (or from underneath the rod or between the rod and bony or other tissue). Even if both rods 34 and 36 have already been placed, and are fixed to vertebrae 62, connector 20 can be used. For example, connector 20 may be maneuvered between rods 34 and 36 and the adjacent tissue so that rod 34 enters channel 26, then connector 20 can be rotated substantially around rod 34 so that rod 36 enters channel 28. With respect to the substantially linear construct embodiment of FIG. 5, connector 20 can essentially make one elongated member out of two. For example, in situations in which support or correction is needed along sections of the spine in which the size of the vertebrae change significantly, as between the cervical and thoracic vertebrae, a larger diameter rod can be connected to relatively lower vertebrae and a smaller diameter rod can be connected to relatively upper vertebrae. Referring to FIG. 5, in that situation rod 36 may have a larger diameter than rod 34, and connector device 20 can connect different diameter rods into essentially one elongated member.

The parts of connector device 20 are composed of biocompatible materials that are also compatible with particular elongated members or other implants with which connector device 20 will be used. Thus, connector device 20 may be made of titanium, nickel, alloys of titanium and nickel, stainless steel, certain sturdy plastic materials, or other sturdy materials. The materials chosen for connector device 20 should be the same as those of the rods with which connector device 20 is used, or at least of a material that will not cause discomfort or an adverse reaction when used with the rods. It will be appreciated that materials other than those described above could also be used.

While the invention has been illustrated and described in detail in the drawings and foregoing description, the same is to be considered as illustrative and not restrictive in character. It should be understood that only the preferred embodiments have been shown and described and that all changes and modifications that come within the spirit of the invention are desired to be protected.

What is claimed is:

1. A connector apparatus for linking elongate members, comprising:
a connector body having a longitudinal axis, a top surface, and a side surface, said body defining a first channel open at said top surface and configured for receipt of a first elongate member, said body further defining a second channel open at said side surface and configured for receipt of a second elongate member, each of said channels defined by an inner contact surface configured to engage said corresponding elongate member;
wherein said first and second channels opening in substantially perpendicular directions;
wherein said first channel is substantially U-shaped and includes an inner threaded portion configured to receive a first threaded retaining member to secure said first elongate member in said first channel; and
wherein said body defines a threaded through-hole from said top surface of said body to said second channel configured for receipt of a second threaded retaining member to secure said second elongate member in said second channel.

2. The apparatus of claim 1, wherein said first channel is positioned to accommodate loading of said first elongate member in a direction perpendicular to said longitudinal axis of said first channel.

3. The apparatus of claim 1, wherein said second channel is positioned to accommodate side loading of said second elongate member in a direction perpendicular to said longitudinal axis of said second channel.

4. The apparatus of claim 1, wherein said second elongate members includes a curved outer surface and said second retaining member includes a curved bearing surface to fittingly contact said curved outer surface of said second elongate member.

5. The apparatus of claim 1, wherein said threaded through-hole and said second threaded retaining member are offset from said second elongate member when said second elongate member is positioned in said second channel.

6. The apparatus of claim 1, wherein said first and second retaining members are operable to advance through said first channel and said through-hole, respectively, to contact said first and second elongate members, respectively, and urge said elongate members against said corresponding inner contact surfaces to engage said connector body to said elongate members.

7. The apparatus of claim 1, wherein first and second retaining members are operable to advance through said first channel and said through-hole, respectively, along directions substantially perpendicular to a longitudinal axis of said connector body.

8. The apparatus of claim 1, wherein each of said elongate members is a spinal rod.

9. The apparatus of claim 1, wherein each of said threaded retaining members is a screw.

10. A connector apparatus for linking spinal rods, comprising:
a connector body having a longitudinal axis and a top surface, said body defining a top opening channel configured for receipt of a first spinal rod and a side opening channel configured for receipt of a second spinal rod, wherein a direction of opening of said first channel is substantially perpendicular to a direction of opening of said second channel;
wherein said top opening channel is U-shaped and includes inner threaded portions configured to engage with a first screw to secure said first spinal rod in said top opening channel;
wherein said body defines a hole from said top surface of said body to said side opening channel, said hole configured for receipt of a second screw to secure said second spinal rod in said side opening channel; and
wherein said hole and said second screw are offset from said second spinal rod when said second spinal rod is positioned in said side opening channel, said second screw being operable to advance through said hole to contact said second spinal rod and push said second spinal rod tightly against an inner contact surface of said side opening channel.

11. The apparatus of claim 10, wherein said hole and said second screw are offset from said second spinal rod in a direction away from said top opening channel.

12. The apparatus of claim 10, wherein first screw advances through said top opening channel, and said second screw advances through said hole, from said top surface of said body.

13. The apparatus of claim 10, wherein said second spinal rod includes a curved outer surface and second screw includes a curved bearing surface to fittingly contact said curved outer surface of said second spinal rod.

14. The apparatus of claim 10, wherein said top opening channel is positioned to accommodate loading of said first spinal rod in a direction perpendicular to said longitudinal axis of said top opening channel.

15. A spinal fixation system, comprising:
first and second spinal rods;
a connector body to connect said spinal rods to each other, said body having a longitudinal axis and a top surface, said body defining a first top opening channel to receive said first spinal rod and a second side opening channel to receive said second spinal rod; and
first and second retaining members to engage said connector body to said corresponding spinal rods, said second retaining member being offset from said second spinal rod, and said first channel including threaded portions to engage with said first retaining member;
wherein said connector body further defines a through-hole in communication with said second channel and configured to receive said second retaining member, wherein said retaining members advance in a direction substantially perpendicular to said longitudinal axis of said connector body, said retaining members being operable to engage said spinal rods against corresponding inner surfaces defining said channels, thereby engaging said connector body to said spinal rods.

16. The system of claim 15, wherein each of said retaining members is a screw.

17. The system of claim 15, wherein said second spinal rod includes an outer curved surface and said second retaining member includes a curved bearing surface configured to contact said outer curved surface of said second spinal rod.

18. The system of claim 15, wherein each of said retaining members includes a recessed hexagonal top portion for receiving an insertion tool, said retaining members being configured to advance below said top surface of said connector body.

19. The system of claim 15, wherein said first channel includes a first direction of opening and said second channel includes a second direction of opening, said first direction of opening being substantially perpendicular to said second direction of opening.

20. A method of linking spinal rods, comprising:
providing a connector apparatus defining a first U-shaped, top opening channel configured for receipt of a first spinal rod and a second side opening channel configured for receipt of a second spinal rod, each of said channels defined by an inner contact surface configured to engage said corresponding spinal rod, said first channel including an inner threaded portion configured to receive a first screw, said apparatus further defining a threaded through-hole in communication with said second channel configured for receipt of a second screw;
positioning said first spinal rod in said first channel;
positioning said second spinal rod in said second channel;
engaging said connector apparatus to said first and second spinal rods; and
connecting one or more of said spinal rods to vertebrae.

21. The method of claim 20, wherein said positioning said first spinal rod includes advancing said apparatus from a position between said first spinal rod and vertebrae, toward said first spinal rod.

22. The method of claim 20, wherein said engaging comprises advancing said first screw through said first channel, and advancing said second screw through said through-hole in communication with said second channel.

23. The method of claim 22, wherein said through-hole and said second screw are offset from said corresponding spinal rod when said spinal rod is positioned in said channel.

24. The method of claim 23, comprising said second screw urging said second spinal rod against said inner contact surface of said second channel to engage said connector apparatus to said second spinal rod.
